(19) United States
(12) Patent Application Publication
(10) Pub. No.: US 2010/0196206 A1
(43) Pub. Date: Aug. 5, 2010

(54) DIAGNOSIS DEVICE USING IMAGE SENSOR AND METHOD OF MANUFACTURING THE SAME

(75) Inventors: Byoung-Su Lee, Yeosu-si (KR); Do-Young Lee, Seongnam-si (KR)

Correspondence Address: Jae Y. Park, Kile, Goekjian, Reed & McManus, PLLC, 1200 New Hampshire Ave. NW, Suite 570, Washington, DC, 20036, US

(73) Assignee: SILICONFILE TECHNOLOGIES INC., Seoul (KR)

(21) Appl. No.: 12/664,812

(22) PCT Filed: Oct. 19, 2007
(86) PCT No.: PCT/KR2007/005147

§ 371 Date: Dec. 15, 2009

(30) Foreign Application Priority Data
Jun. 27, 2007 (KR) 10-2007-0063404

(51) Int. Cl. G01N 33/531; G01N 21/00

(52) U.S. Cl. 422/82.05; 427/162; 216/24; 427/58

(57) ABSTRACT

A diagnosis device using an image sensor and a method of manufacturing the same are provided. The diagnosis device using the image sensor includes: a substrate in which an image sensor including a plurality of optical sensors is formed; an insulation layer formed on the substrate; and a plurality of wells formed in the insulation layer in correspondence with the plurality of optical sensors, the plurality of wells into which reference samples for biochemical reactions with a target sample are inserted.

### BACKGROUND OF THE INVENTION

1. Field of the Invention

The present invention relates to a diagnosis device using an image sensor in which a part in which biochemical reactions occur and a part in which the strength of the biochemical reactions is detected are integrated into a single body.

2. Description of the Related Art

In general, bio-chips have a shape in which reference samples constructed with biological molecules such as DNA, protein, and the like are regularly arranged on a substrate made of glass, silicon, or nylon. The bio-chips are classified into DNA chips and protein chips based on types of reference samples to be arranged. The bio-chips basically use biochemical reactions between reference samples fixed to the substrate and a target sample. The typical example of the biochemical reactions between the reference samples and the target sample may be complementary bonding between DNA bases or antigen-antibody reactions.

A diagnosis using the bio-chip is generally performed by detecting the strength of the biochemical reactions through an optical process. In the generally used optical process, a fluorescent or light emitting characteristic is used.

In an example of the optical process using the fluorescent characteristic, a fluorescent material is mixed with the target sample to be injected into the reference samples fixed in the bio-chip, and the fluorescent material remains in predetermined biochemical reactions between the reference samples and the target sample. Then, the fluorescent material generates light through an external light source, and the generated light is measured.

In an example of the optical process using the light emitting characteristic, a light emitting material is mixed with the target sample to be injected into the reference samples fixed in the bio-chip, the light emitting material remains in predetermined biochemical reactions between the reference samples and the target sample. Then, the light emitting material emits light without an external light source, and the emitted light is measured.

FIG. 1 illustrates a conventional bio-chip.

Referring to FIG. 1, a conventional bio-chip 100 is constructed by arranging various reference samples 120 at a predetermined interval on a substrate 110 made of glass.

When injecting a target sample into the various reference samples 120 in the conventional bio-chip 100, biochemical reactions between the target sample and reference samples 120 occur. At this time, when a predetermined amount of a fluorescent material or light emitting material is included in the target sample through chemical bonding, the fluorescent material or light emitting material remains after the biochemical reactions between the target sample and reference sample 120. When a fluorescent material or light emitting material is generated through the biochemical reactions between the target sample and the reference samples 120, the fluorescent material or light emitting material remains.

The remaining fluorescent material or light emitting material generates light by irradiating the remaining fluorescent material with light or shielding the remaining light emitting material from external light. At this time, since the amount of the fluorescent material or light emitting material is changed based on the strength of the biochemical reactions, the intensity of light generated from the fluorescent material or light emitting material is also changed. In order to measure the intensity of generated light, a separate scanning device such as a CCD camera, a laser scanner, a microscope, and the like is needed. Since the CCD camera, the laser scanner, the microscope, and the like are expensive, it is difficult to commercialize the bio-chip.

FIG. 2 illustrates a CCD camera 210 as an example of an apparatus for scanning a conventional bio-chip.

In general, the intensity of light 202 generated from the fluorescent material by irradiating the fluorescent material with light 201 or the intensity of light generated from the light emitting material by shielding the light emitting material from external light is weak. Accordingly, when a CCD camera 210 is used to sense the light generated from the fluorescent material or light emitting material, since the CCD camera 210 using a semiconductor is weak in thermal noise, a long exposure time is necessary so as to collect light, when the intensity of the light generated from the fluorescent material or light emitting material is weak. Since the thermal noise increases in proportion to the exposure time, the sensed light includes a large amount of noise. Thus, efficiency of sensing light decreases.

In the past, in order to improve the efficiency of sensing light in the CCD camera 210, an expensive lens 211 is added, or the CCD camera 210 is additionally processed. A typical example of the additional process is a process of cooling the CCD camera 210. Since it is possible to reduce the thermal noise generated by thermoelectrons by reducing generation of thermoelectrons by cooling the CCD camera 210. However, a complex procedure for cooling the CCD camera 210 and an additional device is needed.

### SUMMARY OF THE INVENTION

The present invention provides a diagnosis device using an image sensor in which a part in which biochemical reactions occur and a part in which the strength of the biochemical reactions is detected are integrated into a single body.

The present invention also provides a method of manufacturing the diagnosis device using the image sensor by using a general process of manufacturing a semiconductor or using a junction.

According to an aspect of the present invention, there is provided a diagnosis device using an image sensor, the diagnosis device comprising: a substrate in which an image sensor including a plurality of optical sensors is formed; an insulation layer formed on the substrate; and a plurality of hollow wells formed in the insulation layer in correspondence with the plurality of optical sensors.

According to another aspect of the present invention, there is provided a diagnosis device using an image sensor, the diagnosis device comprising: a substrate in which an image sensor including a plurality of optical sensors is formed; a passivation layer formed on the substrate; an insulation layer formed on the passivation layer; and a plurality of hollow wells formed in the insulation layer in correspondence with the plurality of optical sensors.

According to another aspect of the present invention, there is provided a diagnosis device using an image sensor, the diagnosis device comprising: a substrate in which an image sensor including a plurality of optical sensors is formed; a plurality of optical filters formed on the substrate in correspondence with the plurality of optical sensors; an insulation layer formed on the substrate and the plurality of optical filters; and a plurality of hollow wells formed in the insulation layer in correspondence with the plurality of optical sensors.

According to another aspect of the present invention, there is provided a diagnosis device using an image sensor, the diagnosis device comprising: a substrate in which an image sensor including a plurality of optical sensors is formed; and an insulation layer formed on the substrate, wherein reference samples for biochemical reactions with a target sample are located on the insulation layer in correspondence with the plurality of optical sensors.

According to another aspect of the present invention, there is provided a diagnosis device using an image sensor, wherein an upper surface of a first substrate is attached to a lower surface of a second substrate, wherein an image sensor including a plurality of optical sensors are formed in the upper surface of the first substrate, wherein a plurality of hollow wells are formed in an upper surface of the second substrate, and wherein the upper surface of the first substrate is attached to the lower surface of the second substrate, so that the plurality of wells correspond to the plurality of optical sensors.

According to another aspect of the present invention, there is provided a method of manufacturing a diagnosis device, the method comprising: forming an insulation layer on a substrate in which an image sensor including a plurality of optical sensors is formed; and forming a plurality of hollow wells corresponding to the plurality of optical sensors on the insulation layer.

According to another aspect of the present invention, there is provided a method of manufacturing a diagnosis device, the method comprising: forming a plurality of optical filters corresponding to a plurality of optical sensors on a substrate in which an image sensor including the plurality of optical sensors are formed; forming an insulation layer on the substrate and the plurality of optical filters; and forming a plurality of hollow wells corresponding to the plurality of optical sensors in the insulation layer.

According to another aspect of the present invention, there is provided a method of manufacturing a diagnosis device, wherein a lower surface of a second substrate is attached to an upper surface of a first substrate, wherein an image sensor including a plurality of optical sensors are formed in the upper surface of the first substrate, wherein a plurality of hollow wells are formed in an upper surface of the second substrate, and wherein the upper surface of the first substrate is attached to the lower surface of the second substrate, so that the plurality of wells correspond to the plurality of optical sensors.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates a conventional bio-chip;

FIG. 2 illustrates an apparatus for scanning a conventional bio-chip;

FIG. 3 illustrates a diagnosis device using an image sensor according to an embodiment of the present invention;

FIG. 4 illustrates the diagnosis device of FIG. 3 into which a reference samples are inserted;

FIG. 5 illustrates a diagnosis device in which a plurality of optical sensors correspond to a single well;

FIG. 6 illustrates wells having various shapes;

FIG. 7 illustrates an optical shield for a dark reference;

FIG. 8 illustrates a passivation layer formed on a substrate;

FIG. 9 illustrates optical filters on optical sensors;

FIG. 10 illustrates a diagnosis device using an image sensor according to another embodiment of the present invention;

FIG. 11 illustrates an optical filter layer formed on an image sensor;

FIG. 12 illustrates a diagnosis device using an image sensor according to still another embodiment of the present invention;

FIG. 13 illustrates a diagnosis device using an image sensor according to further still another embodiment of the present invention; and

FIG. 14 illustrates a silicon oxide layer formed on a first substrate shown in FIG. 13.

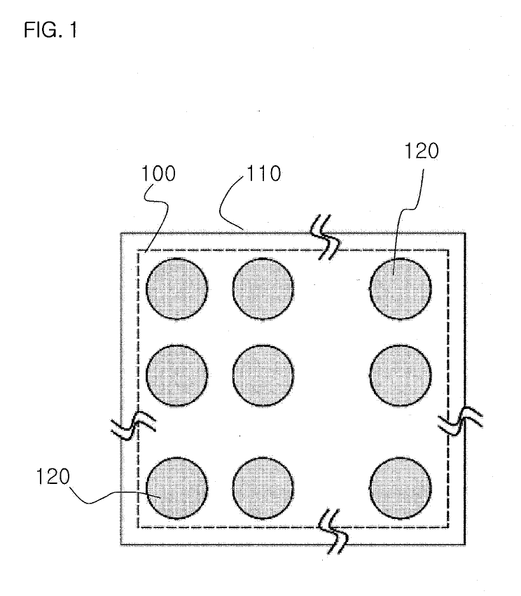

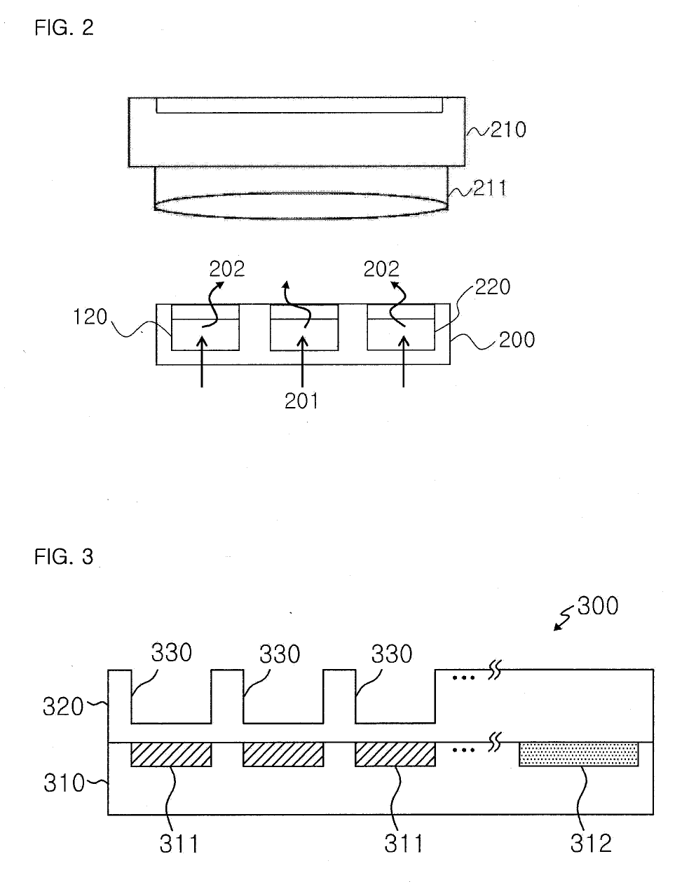

### DETAILED DESCRIPTION OF THE INVENTION

Hereinafter, the present invention will be described in detail with reference to accompanying drawings.

FIG. 3 illustrates a diagnosis device using an image sensor according to an embodiment of the present invention.

A diagnosis device 300 shown in FIG. 3 includes a substrate 310 in which an image sensor is formed, an insulation layer 320, and a plurality of wells 330.

The image sensor including a plurality of optical sensors 311 is formed in the substrate 310. The substrate may be a silicon based substrate typically used in a process of manufacturing a semiconductor. The image sensor may be a widely distributed charge coupled device (CCD) type image sensor or complementary metal oxide semiconductor (CMOS) type image sensor. Since structures and operations of the CMOS type image sensor or CCD type image sensor are well known, detailed description on the image sensor itself will be omitted.

Typical examples of the plurality of optical sensors 311 may be photodiodes or phototransistors. The plurality of optical sensors 311 are formed by doping impurities into the surface of the substrate 310. The plurality of optical sensors 311 sense light and generate charges corresponding to the sensed light. The plurality of optical sensors 311 are connected to peripheral circuits (not shown) for generating signals based on the generated charges. In the CMOS image sensor, the peripheral circuits may be embodied as various circuits including three or four transistors such as transfer transistors and reset transistors.

The insulation layer 320 is formed on the substrate 310 in which the image sensor including the plurality of optical sensors 311 is formed. As will be described, the present invention uses a fluorescent or light emitting phenomenon generated by a fluorescent material or light emitting material remaining after biochemical reactions in the plurality of wells 330. Accordingly, the insulation layer may be transparent. The insulation layer 320 may be made of a glass material such as spin on glass (SOG), undoped silicate glass (USG), phosphor silicate (PSG), boro silicate glass (BSG), boro-phospho silicate glass (BPSG), and low temperature oxide glass (LTO glass).

The plurality of wells 330 are formed in the insulation layer 320 in correspondence with the plurality of optical sensors 311. The plurality of wells 330 are hollow. The insulation layer 320 and the plurality of wells 330 may be easily formed through a deposition process and an etching process in the process of manufacturing a semiconductor. Various reference samples for the biochemical reactions with a target sample are inserted into the plurality of wells 330.

The target sample biochemically reacting with a reference sample in each well 330 may include a light emitting material which emits light by itself, when external light is shielded. In addition, the light emitting material may be formed through a biochemical reaction between the target sample and a reference sample in each well 330. A typical light emitting material may be luciferin. Activated luciferin is formed by activating luciferin by using adenosine tri-phosphate (ATP). The activated luciferin is oxidized by an action of luciferase so as to become luciferin oxide. Thus, chemical energy is changed into optical energy to generate light.

In addition, the target sample reacting with a reference sample in each well 330 may include a fluorescent material such as green fluorescence protein (GFP) that emits light when being irradiated with light. In addition, a fluorescent material may be formed through a biochemical reaction between the target sample and a reference sample in each well 330.

In the diagnosis device 300 shown in FIG. 3, the plurality of wells 330 in which biochemical reactions occur and the plurality of optical sensors 11 are arranged in a single device. Accordingly, it is possible to minimize intervals between the plurality of wells 330 and the optical sensors 311. Thus, it is possible to reduce loss of light in the process of emitting light from the light emitting material or emitting fluorescent light from the fluorescent material remaining after the biochemical reaction in each well 330.

Referring FIG. 3, an image signal processor (ISP) 312 for processing a signal output from the image sensor including the plurality of optical sensors 311 may be further formed in the substrate 310. When the ISP 312 is included in the diagnosis device 300, it is possible to obtain a result of sensing light and a result of processing the result of sensing light based on the result of the biochemical reaction between the target sample and various reference samples in the diagnosis device 300.

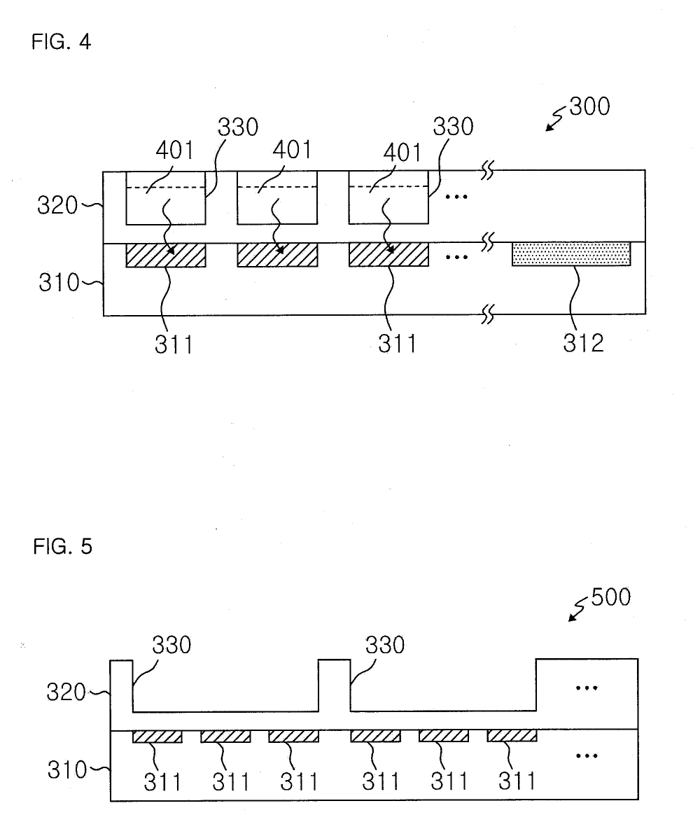

FIG. 4 illustrates the diagnosis device 300 of FIG. 3 in which reference samples 401 are inserted into the plurality of wells 330.

At this time, the reference samples 401 indicate various types of samples for the biochemical reactions with the target sample. The reference samples 401 are changed based on biochemical reactions in the plurality of wells 330 in the diagnosis device 300. When the biochemical reactions are antigen-antibody reactions, the reference samples 401 are antigens. When the biochemical reactions are complementary bonding between DNA bases, the reference samples 401 are genes fabricated for the complementary bonding. The target sample to be biochemically reacted with the reference samples 401 is determined based on the reference samples 401. For example, when the reference samples 401 are antigens, the target sample 401 may be blood. When the reference samples 401 are fabricated genes, the target sample may be a gene of a user.

When the strength of the biochemical reactions between the reference samples 401 and the target sample such as complementary bonding between DNA bases and antigen-antibody reactions is different based on wells 330, the amount of the remaining light emitting material such as luciferin bonded to the target sample is also different. At this time, when external light is shielded so as to allow the remaining light emitting material to emit light, the light emitting material emits different intensity of light in the wells 330 based on the remaining amount of the light emitting material. Accordingly, the intensity of light sensed by the optical sensor 311 corresponding to each well 330 is different from that of light sensed by another optical sensor 311.

FIG. 5 illustrates a diagnosis device 500 in which a plurality of optical sensors 311 correspond to a single well 330. That is, although one optical sensor 311 may be located under a single well 330, a plurality of optical sensors 311 may be arranged under a single well 330 so as to increase reliability of sensing light.

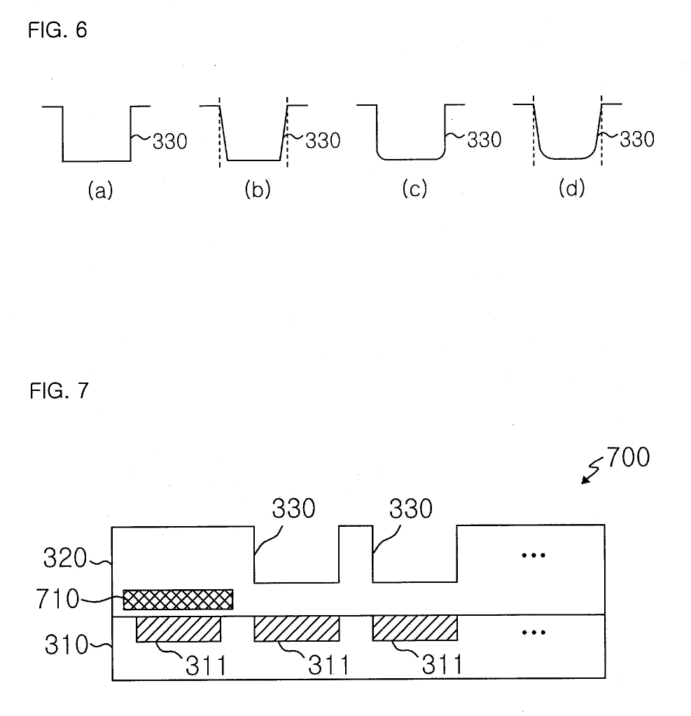

FIG. 6 illustrates wells 300 having various shapes.

Referring to FIG. 6, the plurality of wells 330 have shapes of which upper cross sections are larger than lower cross sections in cases of (a) and (c) or shapes of which upper cross sections are less than lower cross sections in cases of (b) and (d). In addition, the plurality of wells may have shapes with squared edges such as a shape of “└ ┘” shown in (a) and (b) or shapes with rounded edges such as a shape of “∪” shown in (c) and (d).

The various shapes of the wells 330 are different based on methods of forming the wells such as a wet etching method and a dry etching method in the procedure of manufacturing a semiconductor. A shape of a well shown in (a) of FIG. 6 may be formed by using the dry etching method. A shape of a well shown in (b) of FIG. 6 may be formed by using the wet etching method. In addition, a shape of a well shown in (c) of FIG. 6 may be formed by the dry etching method and a reflow method. A shape of a well shown in (d) of FIG. 6 may be formed by the dry etching method, the wet etching method, and a reflow method.

Since a void may be formed in the well with squared edges shown in (a) or (b) of FIG. 6 when inserting reference samples 401 into the well, the shape of the well with rounded edges shown in (c) or (d) of FIG. 6 may be preferable.

FIG. 7 illustrates an optical shielding film 710 for a dark reference.

Referring to a diagnosis device 700 shown in FIG. 7, an optical shielding film 710 may be further formed on at least one of the plurality of optical sensors 311. When the optical shielding film 710 is formed, since light is not incident onto the optical sensors 311 under the optical shielding film 710, the optical sensors 311 may be used as dark references. The optical shielding film 710 may be a metal nitride film such as an aluminum nitride film, a tungsten nitride film, and a titanium nitride film or a black photoresist.

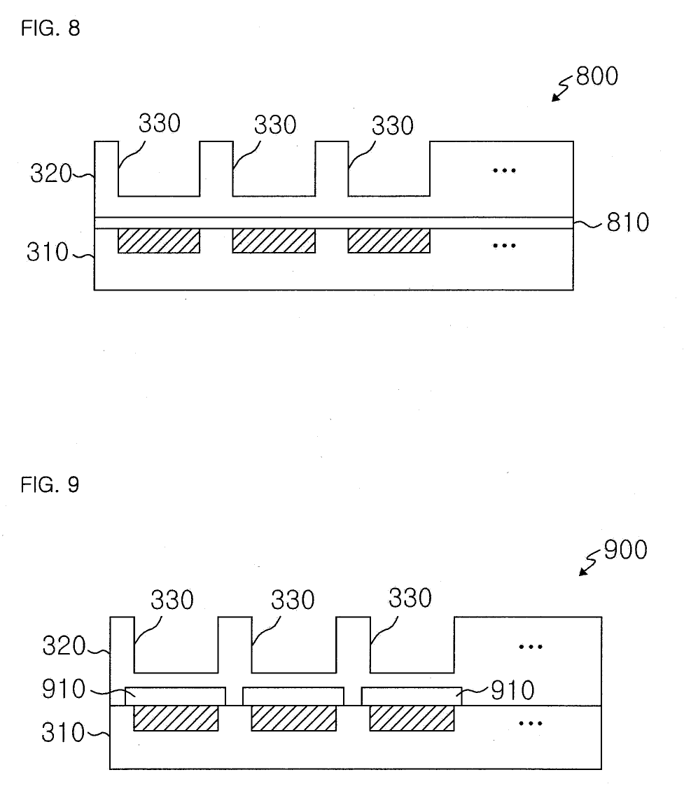

FIG. 8 illustrates a passivation layer 810 formed on a substrate 310.

The passivation layer is generally formed so as to protect semiconductor elements, which are formed before proceeding to the next process after forming the semiconductor elements such as photodiodes in the procedure of manufacturing a semiconductor, from an external impact.

Referring to a diagnosis device 800 shown in FIG. 8, the passivation layer 810 is formed between the substrate 310 in which the image sensor including the plurality of optical sensors 311 is formed and the insulation layer 320 in which the plurality of wells 330 are formed. Here, the passivation layer 810 may be made of a transparent material so as not to prevent light incident onto the plurality of optical sensors 311. Accordingly, the passivation layer 810 may be made of the material that is the same as that of the insulation layer 320. That is, the passivation layer 810 may be made of silicon oxide such as SiO₂, silicon nitride such as Si₃N₄, and glass such as SOG, USG, PSG, BSG, BPSG, and LTO glass.

The material of the insulation layer 320 may be the same as that of the passivation layer 810. This indicates that the insulation layer 320 and the passivation layer 810 may be formed as a single layer.

FIG. 9 illustrates optical filters 910 which are further formed on optical sensors 311.

In general, the optical filters 910 are needed so that only light within a predetermined wavelength band is incident onto the optical sensors 311. When the optical filters 910 are formed on the optical sensors 311, it is possible to increase the efficiency of sensing light in the plurality of optical sensors 311 by preventing light out of the predetermined wavelength band from being incident onto the optical sensors 311. The optical filters 910 may be formed through a spin coating process of the photoresist or an injection process of a metal element such as iron (Fe), copper (Cu), cobalt (Co), manganese (Mn), and antimony (Sb), and the like. In addition, the optical filters 910 may be formed by forming a thin film by changing a deposited material or a thickness of the deposited material by using materials such as silicon dioxide (SiO₂), magnesium fluoride (MgF₂), calcium fluoride (CaF₂), aluminum oxide (Al₂O₃), tin oxide (TiO₂), and the like of which refractive indexes are different from one another with respect to each wavelength.

For example, when a fluorescent material is formed as a result of a biochemical reaction between the reference samples 401 and the target sample, the generated fluorescent material has to be irradiated with light so as to emit light. Blue light or ultraviolet ray is used to irradiate the fluorescent material. Accordingly, the blue light or ultraviolet ray used to irradiate the fluorescent material may be prevented from being incident onto the optical sensors 311. Thus, when the optical filters 910 for allowing only light within the predetermined wavelength band to pass through the optical filters 910 are used, the light used to irradiate the fluorescent material is blocked. Only the light emitted from the fluorescent material is incident onto the optical sensors 311.

Referring to a diagnosis device 900 shown in FIG. 9, the optical filters 910 are formed on the substrate 310 in correspondence with the plurality of optical sensors 311. The insulation layer 320 is formed on the substrate 310 and on the plurality of optical filters 910.

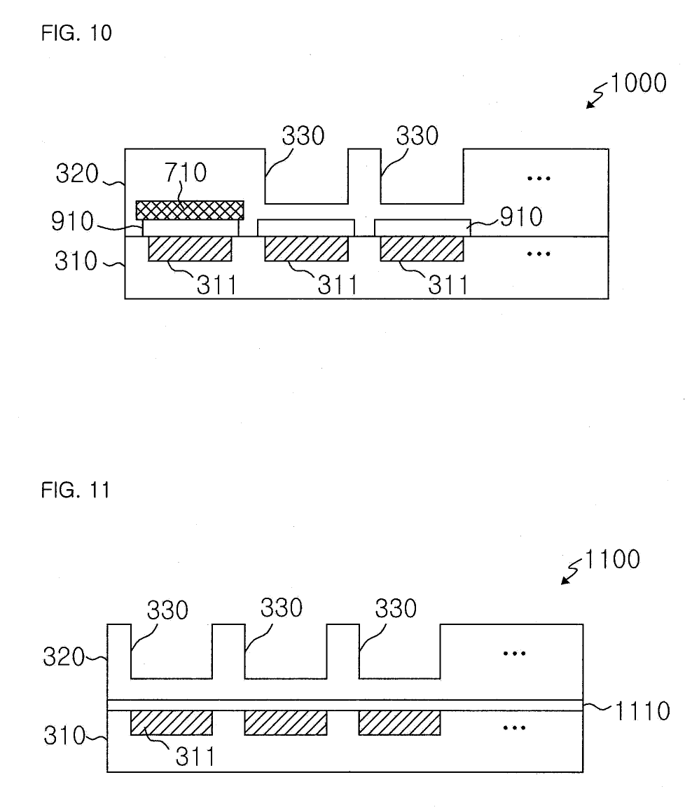

The optical shielding film 710 shown in FIG. 7 may be further formed on or under the at least one of the plurality of optical filters 910. FIG. 10 illustrates a diagnosis device 1000 in which the optical shielding film 710 is formed on an optical filter 910.

The optical filters 910 may be color filters for allowing only light within predetermined wavelength bands corresponding to red (R), green (G), and blue (B) to pass through the optical filters 910. When light of different colors is generated in each well 330, the optical filters 910 are available. When light of different colors is generated in a single well 330, as shown in FIG. 5, the plurality of optical sensors 311 on which the color filters 910 are formed may correspond to a single well 330.

When the plurality of optical filters 910 do not allow light in different wavelength bands to pass through the optical filters 910, an optical filter layer 1110 that is a single layer is formed instead of the plurality of optical filters 910 as in the diagnosis device 1100 shown in FIG. 11.

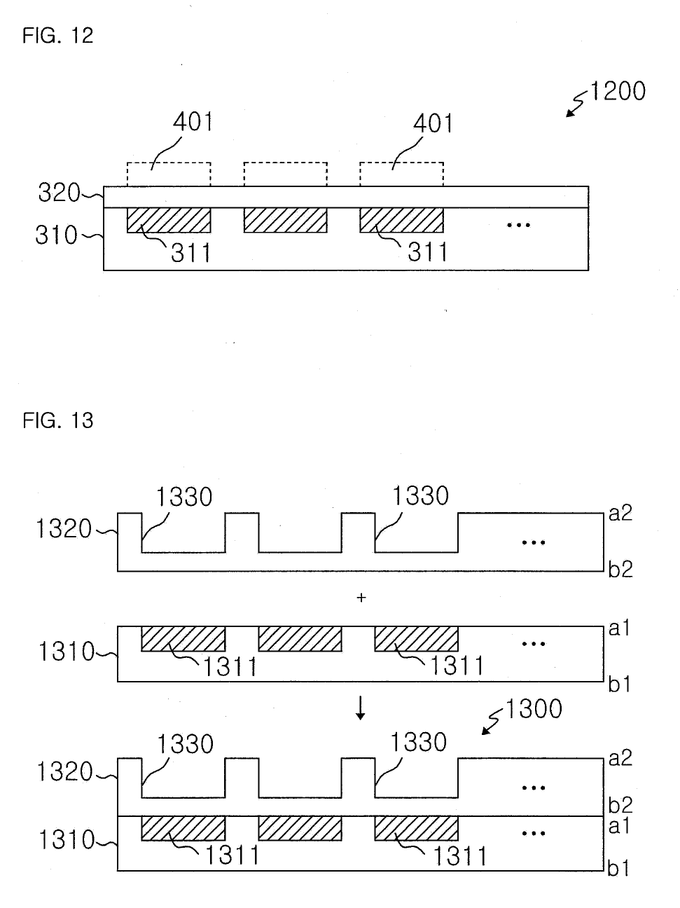

FIG. 12 illustrates a diagnosis device using an image sensor according to still another embodiment of the present invention.

Although the plurality of wells 330 are formed in the insulation layer 320 in FIGS. 3 to 11, wells are not separately formed in the diagnosis device 1200 shown in FIG. 12. The reference samples 401 for biochemical reactions with the target sample are arranged on the insulation layer 320. The reference samples 401 are arranged in correspondence with the plurality of optical sensors 311. In this case, although little interference may occur when the biochemical reactions between the reference samples 401 and the target sample occur, it is possible to easily arrange the reference samples as compared with a case where the reference samples are inserted into the wells 330.

Surely, the diagnosis device 1200 shown in FIG. 12 may further include the optical shielding film 710, the passivation layer 810, and the plurality of optical filters 910, if necessary.

FIG. 13 illustrates a diagnosis device using an image sensor according to further still another embodiment of the present invention.

Referring to FIG. 13, a diagnosis device 1300 is constructed by attaching a lower surface b2 of a second substrate 1320 to an upper surface a1 of a first substrate 1310. An image sensor including a plurality of optical sensors 1311 is formed in the upper surface a1 of the first substrate 1310. A plurality of hollow wells 1330 are formed in an upper surface a2 of the second substrate 1320. At this time, the plurality of wells 1330 correspond to the plurality of optical sensors 1311.

At this time, the first substrate 1310 may be made of silicon. The second substrate 1320 may be made of glass.

The second substrate 1320 may be attached to the first substrate 1310 by using a glass adhesive. Selectively, the second substrate 1320 may be attached to the first substrate 1310 by heating the second substrate 1320. Selectively, the second substrate 1320 may be attached to the first substrate 1310 by using an adhesive polymer such as epoxy. The adhesive polymer may be transparent. When the adhesive polymer has a predetermined color, a color filter having a color that is the same as the predetermined color may be formed on the optical sensors 1311 that are formed on the first substrate 1310.

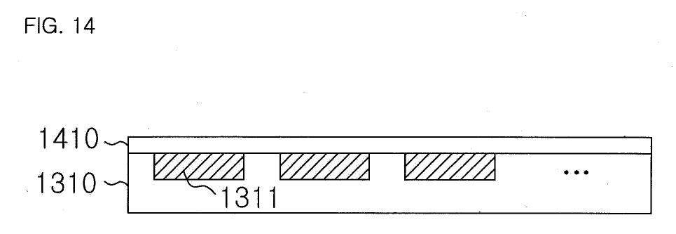

FIG. 14 illustrates a silicon oxide layer formed on the first substrate shown in FIG. 13.

When the second substrate 1320 is made of glass, and when a silicon oxide layer 1410 made of silicon oxide such as SiO₂ is formed on the first substrate 1310, a SiO₂—SiO₂ bonding is formed. Since this is a bonding between the same materials, it is possible to relatively increase bonding efficiency as compared with a case of bonding between different materials. It is possible to use the passivation layer 810 made of silicon oxide or glass not by separately forming the silicon oxide layer 1410. In addition, it is possible to form the silicon oxide layer 1410 on the passivation layer 810 made of a material such as silicon oxide, silicon nitride, glass, and the like.

When the plurality of optical filters 910 corresponding to the plurality of optical sensors 1311 are further formed on the first substrate 1310, the silicon oxide layer 1410 is formed on the first substrate 1310 and on the plurality of optical filters 910.

As described above, in a diagnosis device using an image sensor according to an embodiment of the present invention, since it is possible to minimize an interval between a plurality of wells in which biochemical reactions occur and optical sensors in which strength of the biochemical reactions is detected, it is possible to reduce a loss of light in a procedure of emitting light or generating fluorescent light.

In addition, in the diagnosis device using the image sensor according to an embodiment of the present invention, additional devices such as a separate CCD camera are not necessary.

While the present invention has been particularly shown and described with reference to exemplary embodiments thereof, it will be understood by those skilled in the art that various changes in form and details may be made therein without departing from the spirit and scope of the present invention as defined by the appended claims.

1. A diagnosis device using an image sensor, the diagnosis device comprising:
a substrate in which an image sensor including a plurality of optical sensors is formed;
an insulation layer formed on the substrate; and
a plurality of hollow wells formed in the insulation layer in correspondence with the plurality of optical sensors.

2. The diagnosis device of claim 1, wherein reference samples for biochemical reactions with a target sample are inserted into the plurality of wells.

3. The diagnosis device of claim 1, wherein at least one of the optical sensors is located under each well.

4. The diagnosis device of claim 1, wherein the insulation layer is made of silicon oxide or silicon nitride.

5. The diagnosis device of claim 1, wherein the insulation layer is made of a material selected from the group consisting of SOG (Spin on Glass), USG (Undoped Silicate Glass), PSG (Phospho Silicate Glass), BSG (Boro Silicate Glass), BPSG (Boro-Phospho Silicate Glass) and LTO glass (Low Temperature Oxide Glass).

6. The diagnosis device of claim 1, wherein the plurality of wells have a shape of which the lower cross section is equal to or less than the upper cross section.

7. The diagnosis device of claim 1, wherein the plurality of wells have a shape of “∪” or a shape of “└┘”.

8. The diagnosis device of claim 1, wherein an ISP (image signal processor) for processing a signal output from the image sensor is further formed on the substrate.

9. The diagnosis device of claim 1, wherein an optical shielding film is further formed on at least one of the plurality of optical sensors.

10. A diagnosis device using an image sensor, the diagnosis device comprising:
a substrate in which an image sensor including a plurality of optical sensors is formed;
a passivation layer formed on the substrate;
an insulation layer foamed on the passivation layer; and
a plurality of hollow wells formed in the insulation layer in correspondence with the plurality of optical sensors.

11. The diagnosis device of claim 10, wherein reference samples for biochemical reactions with a target sample are inserted into the plurality of wells.

12. The diagnosis device of claim 10, wherein the passivation layer is made of silicon oxide or silicon nitride.

13. The diagnosis device of claim 10, wherein the passivation layer is made of a material selected from the group consisting of SOG (Spin on Glass), USG (Undoped Silicate Glass), PSG (Phospho Silicate Glass), BSG (Boro Silicate Glass), BPSG (Boro-Phospho Silicate Glass) and LTO glass (Low Temperature Oxide Glass).

14. A diagnosis device using an image sensor, the diagnosis device comprising:
a substrate in which an image sensor including a plurality of optical sensors is formed;
a plurality of optical filters formed on the substrate in correspondence with the plurality of optical sensors;
an insulation layer formed on the substrate and on the plurality of optical filters; and
a plurality of hollow wells formed in the insulation layer in correspondence with the plurality of optical sensors.

15. The diagnosis device of claim 14, wherein reference samples for biochemical reactions with a target sample are inserted into the plurality of wells.

16. The diagnosis device of claim 14, wherein an optical shielding film is further formed on at least one of the plurality of optical filters.

17. The diagnosis device of claim 14, wherein the optical filters are color filters.

18. A diagnosis device using an image sensor, the diagnosis device comprising:
a substrate in which an image sensor including a plurality of optical sensors is foamed;
an optical filter layer formed on the substrate;
an insulation layer formed on the optical filter layer; and
a plurality of hollow wells formed in the insulation layer in correspondence with the plurality of optical sensors.

19. The diagnosis device of claim 18, wherein reference samples for biochemical reactions with a target sample are inserted into the plurality of wells.

20-33. (canceled)

34. A method of manufacturing a diagnosis device, the method comprising:
forming an insulation layer on a substrate in which an image sensor including a plurality of optical sensors is formed; and
forming a plurality of hollow wells corresponding to the plurality of optical sensors on the insulation layer.

35. The method of claim 34, wherein reference samples for biochemical reactions with a target sample are inserted into the plurality of wells.

36. The method of claim 34, wherein the plurality of wells are formed by at least one of methods including a dry etching method and a wet etching method.

37. The method of claim 34, wherein an ISP (image signal processor) for processing a signal output from the image sensor is further formed on the substrate.

38. The method of claim 34, wherein a passivation layer is further formed on the substrate.

39. The method of claim 34, wherein an optical shielding film is further formed on at least one of the plurality of optical sensors.

40. A method of manufacturing a diagnosis device, the method comprising:
forming a plurality of optical filters corresponding to a plurality of optical sensors on a substrate in which an image sensor including the plurality of optical sensors are formed;
forming an insulation layer on the substrate and on the plurality of optical filters; and
forming a plurality of hollow wells corresponding to the plurality of optical sensors in the insulation layer.

41. The method of claim 40, wherein reference samples for biochemical reactions with a target sample are inserted into the plurality of wells.

42-54. (canceled)
